(19) United States
(12) Patent Application Publication
(10) Pub. No.: US 2020/0230031 A1
(43) Pub. Date: Jul. 23, 2020

(54) COMPOSITION

(71) Applicant: COSMETIC WARRIORS LIMITED, Poole, Dorset (GB)

(72) Inventors: Mark CONSTANTINE, Poole, Dorset (GB); Margaret Joan CONSTANTINE, Poole, Dorset (GB); Helen Elizabeth AMBROSEN, Poole, Dorset (GB); Rowena Jacqueline BIRD, Christchurch, Dorset (GB); Jack CONSTNTINE, Poole, Dorset (GB); Anna LEGROS, Poole, Dorset (GB)

(21) Appl. No.: 16/630,354

(22) PCT Filed: Aug. 4, 2017
(86) PCT No.: PCT/GB2017/052302

§ 371 Date: Jan. 10, 2020

(30) Foreign Application Priority Data
Jul. 17, 2017 (GB) 1711446.3

(51) Int. Cl. A61K 8/02; A61K 8/73; A61K 8/365; A61K 8/19

(52) U.S. Cl. CPC A61K 8/0233; A61K 8/733; A61Q 19/10; A61K 8/19; A61K 8/365

(57) ABSTRACT

A solid cosmetic product includes: (i) a first effervescent material, and (ii) a second effervescent material. The second effervescent material contains a salt of alginic acid. Each effervescent material is capable of effervescence on contact with water, with the rate of effervescence of the first effervescent material being greater than the rate of effervescence of the second effervescent material. The first and second effervescent materials are distinct from each other and the second effervescent material envelops the first effervescent material. The solid cosmetic product includes (a) a salt of carbonic acid; (b) an organic acidifying agent; (c) a salt of alginic acid; and (d) a calcium salt.

### FIELD OF THE INVENTION

The present invention relates to a solid cosmetic product, a process for producing said solid cosmetic product, and a product prepared by the process.

### BACKGROUND TO THE INVENTION

The present invention relates to solid cosmetic products, particularly those for use in contact with the human or animal body.

A bath product which has been increasingly popular is the Bath Bomb® or Ballistic®. These products are primarily designed to provide an experience for the user. They typically contain sodium bicarbonate and citric acid such that on contact with water they effervesce to produce carbon dioxide. This effervescence provides a pleasant sensation for the user. The products also typically contain oils and/or fragrances which are liberated with the effervescence. This liberation adds to the sensory experience of the user. Effervescent products have also been used in other application areas. For example, EP1191092 teaches a cleansing product, in particular for cleaning hard surfaces which effervesces in use. To protect the effervescent product from premature activation, the effervescent material is covered with an outer layer which is non-effervescent. This outer layer is formed from a hydrated version of the inner effervescent layer.

Surfactant products such as bubble baths, shampoos, shower gels and toothpastes are extremely well known cosmetic products and personal care products. Bubble bath products are typically provided in the form of liquids. They are sold in containers to the end user and may be dispensed by the end user. However, the required use of packaging is a disadvantage. From an environmental perspective, waste packaging is a significant problem, despite the availability of recycling. For this reason at least, solid materials for creating bath foam have become increasingly popular. These solid surfactant materials may be in the form of a Bubble Bar® For example WO00/47181 discloses a surfactant product bubble bar which is solid and in the form of a tablet or bar. When required for use, a portion of the solid product may be ‘broken off’ the bar and used. The products of WO00/47181 are formed from a composition containing cream of tartar, sodium bicarbonate and a surfactant. Although these products address environmental concerns they suffer certain disadvantages. For example, in use the user must apply the required amount of product to the bath water, for example by holding the solid product under running water. In this way, the required foam is produced. Furthermore, some users may find that they do not provide the same ‘experience’ that a bath ballistic may provide.

The present invention seeks to provide solid cosmetic products which provide the user with an enhanced experience.

### SUMMARY OF THE INVENTION

According to a first aspect of the invention, there is provided a solid cosmetic product comprising (i) a first effervescent material, and (ii) a second effervescent material, the second effervescent material containing a salt of alginic acid, wherein each effervescent material is capable of effervescence on contact with water, wherein the rate of effervescence of the first effervescent material is greater than the rate of effervescence of the second effervescent material, wherein the first and second effervescent materials are distinct from each other, and wherein the second effervescent material envelops the first effervescent material.

According to a second aspect of the invention, there is provided a process for the production of a solid cosmetic product comprising (i) a first effervescent material, and (ii) a second effervescent material, the second effervescent material containing a salt of alginic acid, wherein each effervescent material is capable of effervescence on contact with water, wherein the rate of effervescence of the first effervescent material is greater than the rate of effervescence of the second effervescent material, wherein the first and second effervescent materials are distinct from each other, and wherein the second effervescent material envelops the first effervescent material, the process comprising the steps of:

(i) preparing the first effervescent material;
(ii) enveloping the first effervescent material with the second effervescent material.

According to a third aspect of the invention, there is provided a product obtained or obtainable by a process for the production of a solid cosmetic product comprising (i) a first effervescent material, and (ii) a second effervescent material, the second effervescent material containing a salt of alginic acid, wherein each effervescent material is capable of effervescence on contact with water, wherein the rate of effervescence of the first effervescent material is greater than the rate of effervescence of the second effervescent material, wherein the first and second effervescent materials are distinct from each other, and wherein the second effervescent material envelops the first effervescent material, the process comprising the steps of:

(i) preparing the first effervescent material;
(ii) enveloping the first effervescent material with the second effervescent material.

According to a fourth aspect of the invention, there is provided a solid cosmetic product comprising: (a) a salt of carbonic acid; (b) an organic acidifying agent; (c) a salt of alginic acid; and (d) a calcium salt.

According to a fifth aspect of the invention, there is provided a process for the production of a solid cosmetic product comprising: (a) a salt of carbonic acid; (b) an organic acidifying agent; (c) a salt of alginic acid; and (d) a calcium salt, the process comprising the steps of:

(i) contacting the salt of carbonic acid, organic acidifying agent, salt of alginic acid and calcium salt;
(ii) solidifying the resultant composition.

According to a sixth aspect of the invention, there is provided a product obtained or obtainable by a process for the production of a solid cosmetic product comprising: (a) a salt of carbonic acid; (b) an organic acidifying agent; (c) a salt of alginic acid; and (d) a calcium salt, the process comprising the steps of:

(i) contacting the salt of carbonic acid, organic acidifying agent, salt of alginic acid and calcium salt;
(ii) solidifying the resultant composition

### Advantages

The present inventors surprisingly found that, by providing a solid cosmetic product comprising at least two layers, both of which effervesce on contact with water but at different rates, and wherein one of the effervescent materials comprises an alginate, a cosmetic product is provided that is able to form a gel upon placing the product in water, such as in bathwater. The product does not require action by the user to create the gel and disperse said gel throughout the water other than adding the product to water. This need not be by addition to running water. For example, with the present product, once a bath has been run the product may be added to the water. This may be before or after the user has immersed themselves in the water. Of course, the product could also be added to the bath as the water is running.

The salt of carbonic acid and organic acidifying agent (or other effervescent couple) that may be present in the product then react with water to generate carbon dioxide. This effervescence within the cosmetic product causes dissolution of the product. The alginate contained in the product reacts with the water to form a gel-like substance in the water. As the product effervesces, the gel is dispersed through the water.

It was surprisingly found that, by providing a first effervescent material that is enveloped by the second effervescent material that comprises the alginate, which first effervescent material has a faster rate of effervescence than that of the second effervescent material, the gel formed by the alginate may be more rapidly and evenly dispersed throughout the body of water in which the product is placed. It was found that the inclusion of the alginate in a slower effervescing layer allows for the alginate to be dispersed from the product such that it is pushed away from the core of the cosmetic product. This relatively slow effervescence allows for a more even hydration of the alginate such that an even gel is formed. Without the inclusion of this slower effervescing material, it was found that the alginate tended to not be so evenly dispersed through the water such that non-homogeneous agglomerates of gel formed in the water, which is not desirable for the user. Subsequently, as water gradually dissolves the outer slower effervescing alginate-containing layer, the faster effervescing material may then react with the water to increase the rate of effervescence. This increased effervescence passes through a passage that has been created by the water dissolution of the outer effervescent alginate-containing layer. The faster effervescence passing through this layer pushes the gel formed further away from the core of the cosmetic product, thus enabling a more rapid and even dispersion of the gel formed by the alginate-containing layer throughout the body of water.

Furthermore, by virtue of the different effervescent rates, the product may be found to propel itself within the body of water in which it is placed. This may be found to generate patterns of gel and also to disperse the gel more thoroughly and evenly throughout a large area of the body of water. This enhances the experience of the user.

For ease of reference, the above and further aspects of the present invention are now discussed under appropriate section headings. However, the teachings under each section are not necessarily limited to each particular section.

### DETAILED DESCRIPTION

### Composition

As discussed herein, in a first aspect of the present invention, there is provided a solid cosmetic product comprising (i) a first effervescent material, and (ii) a second effervescent material, the second effervescent material containing a salt of alginic acid, wherein each effervescent material is capable of effervescence on contact with water, wherein the rate of effervescence of the first effervescent material is greater than the rate of effervescence of the second effervescent material, wherein the first and second effervescent materials are distinct from each other, and wherein the second effervescent material envelops the first effervescent material.

It will be understood by one skilled in the art that the nature of a cosmetic product means that the product is not edible. Thus, in some embodiments, the present invention provides a non-edible solid cosmetic product as defined herein.

Solid cosmetic products of the present invention are compositions which can substantially sustain their physical shape when unsupported by external means, e.g. packaging etc. Thus, they are considered to be solid, solid-like, in solid form or in solid-like form at room temperature. For the avoidance of doubt, the solid product is one which remains substantially solid at a temperature of up to 30° C. By solid-like, it is understood that some materials are considered on a day to day basis to be solid, yet over an extremely long period of time, may alter in shape, e.g. amorphous materials such as glass etc. However, they are considered to be solid-like as, for the purpose they fulfil, they are solid.

As mentioned above, due to the solid form of the compositions of the present invention, external packaging is not required to maintain the shape of the composition.

As discussed herein, the first and second effervescent materials are distinct from each other. It will be understood by one skilled in the art that ‘distinct from each other’ requires that the two (or more) materials may not be mixed such that a single combined material results. Furthermore, one skilled in the art will understand that the two materials have different compositions. Without this the rate of effervescence of the first effervescent material would not be greater than the rate of effervescence of the second effervescent material, and the materials would not be distinct.

As discussed herein, the second effervescent material envelops the first effervescent material. As used herein, the term “envelops” means that the second effervescent material substantially covers the first second effervescent material. For example, the second effervescent material covers at least 50% of the outer periphery of the first effervescent material, preferably at least 70% of the outer periphery of the first effervescent material, preferably at least 80% of the outer periphery of the first effervescent material, preferably at least 90% of the outer periphery of the first effervescent material. Preferably the second effervescent material entirely envelops the first effervescent material.

As discussed herein, the second effervescent material (the slower effervescing material) envelops the first effervescent material (the faster effervescing material), and it is preferred that the second effervescent material entirely envelops the first effervescent material. In such aspects it is found that water will gradually dissolve through the outer alginate-containing layer resulting in the initial generation of some gel. When water penetrates to the faster-effervescing first effervescent material then effervescence increases. This increased effervescence passes through a passage that has been created by the water dissolution of the outer effervescent alginate-containing layer. The faster effervescence passing through this layer enables dispersion of the gel formed by the alginate-containing layer through the body of water. Furthermore, if the passage is relatively narrow, the pressure created by the exit of the effervesced gas (carbon dioxide) may propel the cosmetic product in the water in which it is immersed. By this means, the now gel-generating device will move within the body of water, resulting in the gel being dispersed throughout a large area of the body of water. This is highly desirable.

### First Effervescent Material

The first effervescent material may be selected from appropriate materials, which effervesce on contact with water. In some embodiments, the first effervescent material comprises at least a salt of carbonic acid and an organic acidifying agent. These materials are present in any suitable amounts to achieve effervescence. One skilled in the art is able to combine these materials to provide the desired rate of effervescence.

In some embodiments, the salt of carbonic acid is selected from sodium bicarbonate, sodium carbonate, sodium sesquicarbonate, potassium bicarbonate, potassium carbonate, potassium sesquicarbonate, calcium bicarbonate, calcium carbonate, magnesium bicarbonate, magnesium carbonate, ammonium bicarbonate, ammonium carbonate, ammonium sesquicarbonate and mixtures thereof. In some embodiments, the salt of carbonic acid is selected from sodium bicarbonate, sodium carbonate, sodium sesquicarbonate and mixtures thereof. In some preferred embodiments, the salt of carbonic acid is sodium bicarbonate. In some preferred embodiments, the salt of carbonic acid is sodium carbonate.

In some embodiments, the organic acidifying agent is an organic acid and/or diacid. In some embodiments, the organic acidifying agent is selected from tartaric acid, cream of tartar, citric acid, fumaric acid, succinic acid, adipic acid, malic acid, maleic acid, acetic acid, oxalic acid, glutaric acid, malonic acid and mixtures thereof. In some embodiments, the organic acidifying agent is selected from tartaric acid, cream of tartar, citric acid and mixtures thereof. In some preferred embodiments, the organic acidifying agent is citric acid. In some preferred embodiments, the organic acidifying agent is cream of tartar.

In some embodiments the first effervescent material comprises a salt of carbonic acid (such as sodium bicarbonate) in an amount of from about 50 to about 80 wt % based on the first effervescent material. In some embodiments the first effervescent material comprises a salt of carbonic acid (such as sodium bicarbonate) in an amount of from about 55 to about 80 wt % based on the first effervescent material. In some embodiments the first effervescent material comprises a salt of carbonic acid (such as sodium bicarbonate) in an amount of from about 60 to about 80 wt % based on the first effervescent material. In some embodiments the first effervescent material comprises a salt of carbonic acid (such as sodium bicarbonate) in an amount of from about 60 to about 75 wt % based on the first effervescent material. In some embodiments the first effervescent material comprises a salt of carbonic acid (such as sodium bicarbonate) in an amount of from about 65 to about 75 wt % based on the first effervescent material. In some embodiments the first effervescent material comprises a salt of carbonic acid (such as sodium bicarbonate) in an amount of from about 70 to about 75 wt % based on the first effervescent material.

In some embodiments the first effervescent material comprises an organic acidifying agent (such as citric acid) in an amount of from about 15 to about 30 wt % based on the first effervescent material. In some embodiments the first effervescent material comprises an organic acidifying agent (such as citric acid) in an amount of from about 20 to about 30 wt % based on the first effervescent material. In some embodiments the first effervescent material comprises an organic acidifying agent (such as citric acid) in an amount of from about 22 to 28 wt % based on the first effervescent material. In some embodiments the first effervescent material comprises an organic acidifying agent (such as citric acid) in an amount of from about 24 to about 27 wt % based on the first effervescent material. In some embodiments the first effervescent material comprises an organic acidifying agent (such as citric acid) in an amount of approximately 25 wt % based on the first effervescent material.

In some embodiments the first effervescent material comprises a salt of carbonic acid (such as sodium bicarbonate) in an amount of from about 50 to about 80 wt % based on the first effervescent material, and an organic acidifying agent (such as citric acid) in an amount of from about 15 to about 30 wt % based on the first effervescent material. In some embodiments the first effervescent material comprises a salt of carbonic acid (such as sodium bicarbonate) in an amount of from about 60 to about 75 wt % based on the first effervescent material, and an organic acidifying agent (such as citric acid) in an amount of from about 20 to about 30 wt % based on the first effervescent material.

In some preferred the first effervescent material comprises sodium bicarbonate in an amount of from about 50 to about 80 wt % based on the first effervescent material. In some preferred embodiments the first effervescent material comprises citric acid in an amount of from about 15 to about 30 wt % based on the first effervescent material.

In some embodiments the first effervescent material comprises a sodium bicarbonate in an amount of from about 50 to about 80 wt % based on the first effervescent material, and citric acid in an amount of from about 15 to about 30 wt % based on the first effervescent material.

In some embodiments the first effervescent material is substantially free of surfactant. By substantially free of surfactant it is meant the first effervescent material comprises surfactant in an amount of less than 1 wt % based on the first effervescent material, such as in an amount of less than 0.5 wt % based on the first effervescent material, such as in an amount of less than 0.1 wt % based on the first effervescent material, such as in an amount of less than 0.01 wt % based on the first effervescent material, such as in an amount of less than 0.001 wt % based on the first effervescent material, such as in an amount of less than 0.001 wt % based on the first effervescent material. In some embodiments the first effervescent material is free of surfactant.

In some embodiments, the first effervescent material comprises a calcium salt. Preferably, the calcium salt is not one which is dissolved in water. Preferably, the calcium salt is in powder form. In some embodiments, the calcium salt (preferably in powder form) is selected from calcium lactate, calcium chloride, calcium gluconate, calcium lactate gluconate, and mixtures thereof. In some embodiments, the first effervescent material comprises calcium lactate.

In some embodiments in which the first effervescent material comprises a calcium salt, the calcium salt is present in an amount of from about 0.1 to about 20 wt % based on the first effervescent material. In some embodiments in which the first effervescent material comprises a calcium salt, the calcium salt is present in an amount of from about 5 to about 15 wt % based on the first effervescent material. In some embodiments in which the first effervescent material comprises a calcium salt, the calcium salt is present in an amount of from about 8 to about 12 wt % based on the first effervescent material.

### Second Effervescent Material

The second effervescent material may be selected from appropriate materials which effervesce on contact with water. In some embodiments the second effervescent material comprises at least a salt of carbonic acid and an organic acidifying agent. These materials are present in any suitable amounts to achieve effervescence. One skilled in the art is able to combine these materials to provide the desired rate of effervescence.

In some embodiments, the salt of carbonic acid is selected from sodium bicarbonate, sodium carbonate, sodium sesquicarbonate, potassium bicarbonate, potassium carbonate, potassium sesquicarbonate, calcium bicarbonate, calcium carbonate, magnesium bicarbonate, magnesium carbonate, ammonium bicarbonate, ammonium carbonate, ammonium sesquicarbonate and mixtures thereof. In some embodiments, the salt of carbonic acid is selected from sodium bicarbonate, sodium carbonate, sodium sesquicarbonate and mixtures thereof. In some preferred embodiments, the salt of carbonic acid is sodium bicarbonate. In some preferred embodiments, the salt of carbonic acid is sodium carbonate.

In some embodiments, the organic acidifying agent is an organic acid and/or diacid. In some embodiments, the organic acidifying agent is selected from tartaric acid, cream of tartar, citric acid, fumaric acid, succinic acid, adipic acid, malic acid, maleic acid, acetic acid, oxalic acid, glutaric acid, malonic acid and mixtures thereof. In some embodiments, the organic acidifying agent is selected from tartaric acid, cream of tartar, citric acid and mixtures thereof. In some preferred embodiments, the organic acidifying agent is citric acid. In some preferred embodiments, the organic acidifying agent is cream of tartar.

In some embodiments the second effervescent material comprises a salt of carbonic acid (such as sodium bicarbonate) in an amount of from about 35 to about 65 wt % based on the second effervescent material. In some embodiments the second effervescent material comprises a salt of carbonic acid (such as sodium bicarbonate) in an amount of from about 35 to about 60 wt % based on the second effervescent material. In some embodiments the second effervescent material comprises a salt of carbonic acid (such as sodium bicarbonate) in an amount of from about 40 to about 60 wt % based on the second effervescent material. In some embodiments the second effervescent material comprises a salt of carbonic acid (such as sodium bicarbonate) in an amount of from about 45 to about 60 wt % based on the second effervescent material. In some embodiments the second effervescent material comprises a salt of carbonic acid (such as sodium bicarbonate) in an amount of from about 45 to about 55 wt % based on the second effervescent material.

In some embodiments the second effervescent material comprises an organic acidifying agent (such as citric acid) in an amount of from about 15 to about 30 wt % based on the second effervescent material. In some embodiments the second effervescent material comprises an organic acidifying agent (such as citric acid) in an amount of from about 20 to about 30 wt % based on the second effervescent material. In some embodiments the second effervescent material comprises an organic acidifying agent (such as citric acid) in an amount of from about 22 to 28 wt % based on the second effervescent material. In some embodiments the second effervescent material comprises an organic acidifying agent (such as citric acid) in an amount of from about 24 to about 27 wt % based on the second effervescent material. In some embodiments the second effervescent material comprises an organic acidifying agent (such as citric acid) in an amount of approximately 25 wt % based on the second effervescent material.

In some embodiments the second effervescent material comprises a salt of carbonic acid (such as sodium bicarbonate) in an amount of from about 35 to about 60 wt % based on the second effervescent material, and an organic acidifying agent (such as citric acid) in an amount of from about 15 to about 30 wt % based on the second effervescent material. In some embodiments the second effervescent material comprises a salt of carbonic acid (such as sodium bicarbonate) in an amount of from about 45 to about 55 wt % based on the second effervescent material, and an organic acidifying agent (such as citric acid) in an amount of from about 20 to about 30 wt % based on the second effervescent material.

In some preferred the second effervescent material comprises sodium bicarbonate in an amount of from about 35 to about 60 wt % based on the second effervescent material. In some preferred embodiments the second effervescent material comprises citric acid in an amount of from about 15 to about 30 wt % based on the second effervescent material.

In some embodiments the second effervescent material comprises sodium bicarbonate in an amount of from about 35 to about 60 wt % based on the second effervescent material, and citric acid in an amount of from about 15 to about 30 wt % based on the second effervescent material.

As discussed herein, the second effervescent material comprises a salt of alginic acid. In some embodiments, the salt of alginic acid comprises monovalent cations. In some embodiments, the salt of alginic acid comprises divalent cations. In some embodiments, the salt of alginic acid is selected from sodium alginate, potassium alginate, ammonium alginate, calcium alginate, and mixtures thereof. In some preferred embodiments, the salt of alginic acid is sodium alginate.

In some embodiments, the second effervescent material comprises the salt of alginic acid (such as sodium alginate) in an amount of no greater than about 30 wt %, such as in an amount of from about 1 to about 30 wt % based on the second effervescent material, such as in an amount of from about 1 to about 25 wt % based on the second effervescent material. In some embodiments the second effervescent material comprises the salt of alginic acid (such as sodium alginate) in an amount of from about 5 to about 25 wt % based on the second effervescent material. In some embodiments the second effervescent material comprises the salt of alginic acid (such as sodium alginate) in an amount of from about 10 to about 20 wt % based on the second effervescent material.

In some embodiments, the second effervescent material further comprises a calcium salt. Preferably, the calcium salt is not one which is dissolved in water. Preferably, the calcium salt is in powder form. In some embodiments, the calcium salt (preferably in powder form) is selected from calcium lactate, calcium chloride, calcium gluconate, calcium lactate gluconate, and mixtures thereof. In some embodiments, the second effervescent material further comprises calcium lactate.

In some embodiments in which the second effervescent material comprises a calcium salt, the calcium salt (such as calcium lactate) is present in an amount of from about 0.1 to about 20 wt % based on the second effervescent material. In some embodiments in which the second effervescent material comprises a calcium salt, the calcium salt (such as calcium lactate) is present in an amount of from about 5 to about 15 wt % based on the second effervescent material. In some embodiments in which the second effervescent material comprises a calcium salt, the calcium salt (such as calcium lactate) is present in an amount of from about 8 to about 12 wt % based on the second effervescent material.

In some embodiments, the second effervescent material comprises a salt of alginic acid and a calcium salt in a weight ratio of from about 10:1 to about 1:10, such as from about 5:1 to about 1:5, such as from about 2:1 to about 1:2, such as approximately 1:1.

In some embodiments, the second effervescent material comprises sodium alginate and calcium lactate in a weight ratio of from about 10:1 to about 1:10, such as from about 5:1 to about 1:5, such as from about 2:1 to about 1:2, such as approximately 1:1.

In some embodiments, the second effervescent material comprises a salt of alginic acid in an amount of from about 5 to about 15 wt % based on the second effervescent material, and a calcium salt in an amount of from about 5 to about 15 wt %. In some preferred embodiments, the second effervescent material comprises sodium alginate in an amount of from about 5 to about 15 wt % based on the second effervescent material, and calcium lactate in an amount of from about 5 to about 15 wt % based on the second effervescent material.

In some embodiments, the second effervescent material comprises:
(i) an organic acidifying agent in an amount of from about 15 to about 30 wt % based on the second effervescent material;
(ii) a salt of carbonic acid in an amount of from about 35 to about 60 wt % based on the second effervescent material; and
(iii) a salt of alginic acid in an amount of from about 1 to about 25 wt % based on the second effervescent material.

In some embodiments, the second effervescent material comprises:
(i) citric acid in an amount of from about 15 to about 30 wt % based on the second effervescent material;
(ii) sodium bicarbonate in an amount of from about 35 to about 60 wt % based on the second effervescent material; and
(iii) sodium alginate in an amount of from about 1 to about 30 wt % based on the second effervescent material.

In some embodiments, the second effervescent material comprises:
(i) an organic acidifying agent in an amount of from about 20 to about 30 wt % based on the second effervescent material;
(ii) a salt of carbonic acid in an amount of from about 45 to about 55 wt % based on the second effervescent material; and
(iii) a salt of alginic acid in an amount of from about 10 to about 20 wt % based on the second effervescent material.

In some embodiments, the second effervescent material comprises:
(i) citric acid in an amount of from about 20 to about 30 wt % based on the second effervescent material;
(ii) sodium bicarbonate in an amount of from about 45 to about 55 wt % based on the second effervescent material; and
(iii) sodium alginate in an amount of from about 10 to about 20 wt % based on the second effervescent material.

In some embodiments, the second effervescent material comprises:
(i) an organic acidifying agent in an amount of from about 15 to about 30 wt % based on the second effervescent material;
(ii) a salt of carbonic acid in an amount of from about 35 to about 55 wt % based on the second effervescent material;
(iii) a salt of alginic acid in an amount of from about 1 to about 25 wt % based on the second effervescent material; and
(iv) a calcium salt in an amount of from about 0.1 to about 20 wt % based on the second effervescent material.

In some embodiments, the second effervescent material comprises:
(i) citric acid in an amount of from about 15 to about 30 wt % based on the second effervescent material;
(ii) sodium bicarbonate in an amount of from about 35 to about 55 wt % based on the second effervescent material;
(iii) sodium alginate in an amount of from about 1 to about 25 wt % based on the second effervescent material; and
(iv) calcium lactate in an amount of from about 0.1 to about 20 wt % based on the second effervescent material.

In some embodiments, the second effervescent material comprises:
(i) an organic acidifying agent in an amount of from about 20 to about 30 wt % based on the second effervescent material;
(ii) a salt of carbonic acid in an amount of from about 45 to about 55 wt % based on the second effervescent material;
(iii) a salt of alginic acid in an amount of from about 10 to about 20 wt % based on the second effervescent material; and
(iv) a calcium salt in an amount of from about 5 to about 15 wt % based on the second effervescent material.

In some embodiments, the second effervescent material comprises:
(i) citric acid in an amount of from about 20 to about 30 wt % based on the second effervescent material;
(ii) sodium bicarbonate in an amount of from about 45 to about 55 wt % based on the second effervescent material;
(iii) sodium alginate in an amount of from about 10 to about 20 wt % based on the second effervescent material; and
(iv) calcium lactate in an amount of from about 15 to about 15 wt % based on the second effervescent material.

### Further Effervescent Materials

The present invention is not limited to only two distinct effervescent materials. A further, third effervescent material may be incorporated into the product to provide further effects, for example with regard to effervescence, colour or gel production.

In some preferred embodiments, the solid cosmetic product further comprises a third effervescent material that is capable of effervescence on contact with water, wherein the third effervescent material is distinct from the first and second effervescent materials.

In some embodiments, the rate of effervescence of the second effervescent material is greater than the rate of effervescence of the third effervescent material. Therefore, in these embodiments, the rate of effervescence of the first effervescent material is greater than the rate of effervescence of the second effervescent material, which in turn is greater than the rate of effervescence of the third effervescent material. In these embodiments, the third effervescent material has the lowest rate of effervescence of the three effervescent materials.

In some embodiments, the rate of effervescence of the third effervescent material is greater than the rate of the second effervescent material. In some embodiments the rate of effervescence of the third effervescent material is greater than the rate of effervescence of the first effervescent material. In some embodiments the rate of effervescence of the third effervescent material is less than the rate of effervescence of the first effervescent material. In some embodiments the rate of effervescence of the third effervescent material is approximately the same as the rate of effervescence of the first effervescent material. In these embodiments, the second effervescent material has the lowest rate of effervescence of the three effervescent materials.

Preferably, the third effervescent material is in one or more distinct pieces disposed at the surface of the second effervescent material. Preferably, the third effervescent material is in multiple pieces disposed at the surface of the second effervescent material. Alternatively, the third effervescent material may be provided in one single piece that is disposed at the surface of the second effervescent material; for example, the third effervescent material may at least partially envelop the second effervescent material. In some embodiments, the third effervescent material fully envelops the second effervescent material. As used herein, “disposed at the surface of the second effervescent material” means that the third effervescent material is adhered to the outside surface of the second effervescent material, or is partially embedded therein such that the third effervescent material is not completely covered by the second effervescent material.

In some embodiments the third effervescent material comprises at least a salt of carbonic acid and an organic acidifying agent.

In some embodiments, the salt of carbonic acid is selected from sodium bicarbonate, sodium carbonate, sodium sesquicarbonate, potassium bicarbonate, potassium carbonate, potassium sesquicarbonate, calcium bicarbonate, calcium carbonate, magnesium bicarbonate, magnesium carbonate, ammonium bicarbonate, ammonium carbonate, ammonium sesquicarbonate and mixtures thereof. In some embodiments, the salt of carbonic acid is selected from sodium bicarbonate, sodium carbonate, sodium sesquicarbonate and mixtures thereof. In some preferred embodiments, the salt of carbonic acid is sodium bicarbonate. In some preferred embodiments, the salt of carbonic acid is sodium carbonate.

In some embodiments, the organic acidifying agent is an organic acid and/or diacid. In some embodiments, the organic acidifying agent is selected from tartaric acid, cream of tartar, citric acid, fumaric acid, succinic acid, adipic acid, malic acid, maleic acid, acetic acid, oxalic acid, glutaric acid, malonic acid and mixtures thereof. In some embodiments, the organic acidifying agent is selected from tartaric acid, cream of tartar, citric acid and mixtures thereof. In some preferred embodiments, the organic acidifying agent is citric acid. In some preferred embodiments, the organic acidifying agent is cream of tartar.

In some embodiments, the third effervescent material comprises a salt of carbonic acid (such as sodium bicarbonate) in an amount of from about 50 to about 80 wt % based on the third effervescent material, such as in an amount of from about 55 to about 80 wt % based on the third effervescent material, such as in an amount of from about 60 to about 75 wt % based on the third effervescent material, such as in an amount of from about 65 to about 75 wt % based on the third effervescent material. In some embodiments, the third effervescent material comprises a salt of carbonic acid (such as sodium bicarbonate) in an amount of from about 65 to about 70 wt % based on the third effervescent material. In some embodiments, the third effervescent material comprises a salt of carbonic acid (such as sodium bicarbonate) in an amount of from about 70 to about 75 wt % based on the third effervescent material.

In some embodiments, the third effervescent material comprises an organic acidifying agent (such as citric acid) in an amount of from about 15 to about 30 wt % based on the third effervescent material, such as in an amount of from about 20 to about 30 wt % based on the third effervescent material, such as in an amount of from about 20 to about 25 wt % based on the third effervescent material.

In some embodiments, the composition of the third effervescent material is substantially the same as the composition of the first effervescent material (i.e. the first and third effervescent materials are distinct, and yet comprise substantially the same components in substantially the same amounts). In some embodiments, the composition of the third effervescent material is the same as the composition of the first effervescent material. In such embodiments, the rate of effervescence of the third effervescent material is greater than the rate of effervescence of the second effervescent material.

In some embodiments the third effervescent material of the solid cosmetic product comprises a surfactant. The surfactant may primarily be selected from those surfactants known in the art to be suitable for contact with the skin. In some embodiments, the surfactant is selected from the group consisting of sodium laureth sulfate, cocamide diethanolamine, lauryl betaine, laureth-4 and mixtures thereof. In some embodiments, the surfactant is sodium laureth sulfate. In some embodiments, the surfactant is lauryl betaine. In some embodiments the surfactant is a mixture of sodium laureth sulfate and lauryl betaine.

In some embodiments the third effervescent material comprises surfactant (such as a mixture of sodium laureth sulfate and lauryl betaine) in an amount of from about 0.01 to about 5 wt % based on the third effervescent material, such as in an amount of from about 0.1 to about 3 wt % based on the third effervescent material, such as in an amount of from about 0.5 to about 2.5 wt % based on the third effervescent material.

In some embodiments, the third effervescent material of the solid cosmetic product comprises cream of tartar in addition to the organic acidifying agent. In some embodiments, the third effervescent material comprises cream of tartar in addition to the organic acidifying agent in an amount of from about 0.01 to about 5 wt % based on the third effervescent material, such as in an amount of from about 0.1 to about 4 wt % based on the third effervescent material, such as in an amount of from about 0.5 to about 3 wt % based on the third effervescent material, such as in an amount of from about 1 to about 2 wt % based on the third effervescent material.

It was surprisingly found that, when a third effervescent material is provided, the gel produced by the alginate in the second effervescent material is more efficiently dispersed throughout the body of water in which the product is placed. In particular, when the third effervescent material is disposed at the surface of the second effervescent material in one or more distinct pieces, the third effervescent material may help to initially disperse the gel formed by the alginate in the second effervescent material through the water.

Subsequently, when water penetrates through the second effervescent material layer, the first effervescent material will effervesce more rapidly, which pushes the gel formed further throughout the water. Furthermore, the rapid effervescence produced by the first effervescent material may propel the product through the water leading to efficient dispersion of the gel throughout the body of water.

### Solid Cosmetic Product

The first effervescent material and the second effervescent material are combined to prepare a solid cosmetic product in accordance with the present invention. The solid cosmetic product may of course contain components in addition to the first effervescent material and the second effervescent material. However, in some embodiments the surfactant product consists of the first effervescent material and the second effervescent material.

The first effervescent material and the second effervescent material may be combined in any suitable ratio to provide the desired solid cosmetic product. Preferred ratios (based on weight) of first effervescent material to second effervescent material are 90:10 to 10:90, 80:20 to 20:80, 70:30 to 30:70, 60:40 to 40:60, 55:45 to 45:55 and approximately 50:50. In alternative embodiments the ratio (based on weight) of first effervescent material to second effervescent material is from about 90:10 to about 50:50, such as from about 80:20 to about 60:40, such as from about 70:30 to about 60:40.

One skilled in the art can readily prepare a product based on these ratios and the amounts of materials based on the first and second effervescent materials.

In some embodiments, the first effervescent material is present in an amount of from about 10 to about 60 wt % based on the solid cosmetic product, such as in an amount of from about 15 to about 50 wt % based on the solid cosmetic product, such as in an amount of from about 20 to about 40 wt % based on the solid cosmetic product, such as in an amount of from about 20 to about 35 wt % based on the solid cosmetic product. In some embodiments, the first effervescent material is present in an amount of from about 20 to about 30 wt % based on the solid cosmetic product.

In some embodiments, the second effervescent material is present in an amount of from about 20 to about 90 wt % based on the solid cosmetic product, such as in an amount of from about 20 to about 85 wt % based on the solid cosmetic product, such as in an amount of from about 30 to about 85 wt % based on the solid cosmetic product, such as in an amount of from about 40 to about 85 wt % based on the solid cosmetic product, such as in an amount of from about 50 to about 85 wt % based on the solid cosmetic product, such as in an amount of from about 60 to about 80 wt % based on the solid cosmetic product. In some embodiments, the second effervescent material is present in an amount of from about 60 to about 70 wt % based on the solid cosmetic product.

In some embodiments the first effervescent material comprises a salt of carbonic acid (such as sodium bicarbonate) in an amount of from about 5 to about 20 wt % based on the solid cosmetic product, such as in an amount of from about 10 to about 20 wt % based on the solid cosmetic product. In some embodiments the first effervescent material comprises an organic acidifying agent (such as citric acid) in an amount of from about 1 to about 10 wt % based on the solid cosmetic product, such as in an amount of from about 5 to about 7 wt % based on the solid cosmetic product.

In some embodiments the second effervescent material comprises a salt of carbonic acid (such as sodium bicarbonate) in an amount of from about 20 to about 50 wt % based on the solid cosmetic product, such as in amount of from about 30 to about 40 wt % based on the solid cosmetic product. In some embodiments the second effervescent material comprises an organic acidifying agent (such as citric acid) in an amount of from about 5 to about 25 wt % based on the solid cosmetic product, such as in an amount of from about 10 to about 20 wt % based on the solid cosmetic product. In some embodiments the second effervescent material comprises a salt of alginic acid (such as sodium alginate) in an amount of from about 0.5 to about 25 wt % based on the solid cosmetic product, such as in an amount of from about 1 to about 20 wt % based on the solid cosmetic product, such as in an amount of from about 5 to about 15 wt % based on the solid cosmetic product. In some embodiments the second effervescent material comprises a calcium salt (such as calcium lactate) in an amount of from about 0.5 to about 15 wt % based on the solid cosmetic product, such as in an amount of from about 1 to about 10 wt % based on the solid cosmetic product.

In some preferred embodiments the first effervescent material comprises a salt of carbonic acid (such as sodium bicarbonate) in an amount of from about 5 to about 20 wt % based on the solid cosmetic product, the first effervescent material comprises an organic acidifying agent (such as citric acid) in an amount of from about 1 to about 10 wt % based on the solid cosmetic product, the second effervescent material comprises a salt of carbonic acid (such as sodium bicarbonate) in an amount of from about 20 to about 50 wt % based on the solid cosmetic product, the second effervescent material comprises an organic acidifying agent (such as citric acid) in an amount of from about 5 to about 25 wt % based on the solid cosmetic product and the second effervescent material comprises a salt of alginic acid (such as sodium alginate) in an amount of from about 0.5 to about 25 wt % based on the solid cosmetic product.

The components of the solid cosmetic product may be selected such that the final product has a density of less than 1 g/cm³, and therefore floats in water, or greater than 1 g/cm³, and therefore sinks in water. A product which floats will tend to move across the surface of the water. This will produce patterns of gel and bubbles as described herein. A product that sinks will tend to provide a ‘geyser’ or ‘whirlpool’ effect. Preferably the solid cosmetic product has a density of less than 1 g/cm³.

As noted above, in some embodiments, the solid cosmetic product further comprises a third effervescent material that is capable of effervescence on contact with water, wherein the third effervescent material is distinct from the first and second effervescent materials. In these embodiments, the third effervescent material is as defined above. In some embodiments, the solid cosmetic product may comprise the third effervescent material in an amount of from about 1 to about 30 wt % based on the solid cosmetic product. In some embodiments, the solid cosmetic product may comprise the third effervescent material in an amount of from about 1 to about 20 wt % based on the solid cosmetic product. In some embodiments, the solid cosmetic product may comprise the third effervescent material in an amount of from about 5 to about 15 wt % based on the solid cosmetic product. In some embodiments, the solid cosmetic product may comprise the third effervescent material in an amount of from about 10 to about 15 wt % based on the solid cosmetic product.

In some embodiments the solid cosmetic product comprises:
(i) the first effervescent material in an amount of from about 10 to about 60 wt % based on the solid cosmetic product;
(ii) the second effervescent material in an amount of from about 20 to about 85 wt % based on the solid cosmetic product; and
(iii) the third effervescent material in an amount of from about 5 to about 30 wt % based on the solid cosmetic product.

In some embodiments the solid cosmetic product comprises:
(i) the first effervescent material in an amount of from about 15 to about 35 wt % based on the solid cosmetic product;
(ii) the second effervescent material in an amount of from about 60 to about 80 wt % based on the solid cosmetic product; and
(iii) the third effervescent material in an amount of from about 5 to about 20 wt % based on the solid cosmetic product.

In some embodiments the third effervescent material comprises a salt of carbonic acid (such as sodium bicarbonate) in an amount of from about 5 to about 10 wt % based on the solid cosmetic product, such as in an amount of from about 6 to about 8 wt % based on the solid cosmetic product. In some embodiments the third effervescent material comprises an organic acidifying agent (such as citric acid) in an amount of from about 1 to about 5 wt % based on the solid cosmetic product, such as in an amount of from about 2 to about 4 wt % based on the solid cosmetic product.

In some preferred embodiments the first effervescent material comprises a salt of carbonic acid (such as sodium bicarbonate) in an amount of from about 5 to about 20 wt % based on the solid cosmetic product, the first effervescent material comprises an organic acidifying agent (such as citric acid) in an amount of from about 1 to about 10 wt % based on the solid cosmetic product, the second effervescent material comprises a salt of carbonic acid (such as sodium bicarbonate) in an amount of from about 20 to about 50 wt % based on the solid cosmetic product, the second effervescent material comprises an organic acidifying agent (such as citric acid) in an amount of from about 5 to about 25 wt % based on the solid cosmetic product, the second effervescent material comprises a salt of alginic acid (such as sodium alginate) in an amount of from about 0.5 to about 25 wt % based on the solid cosmetic product, the third effervescent material comprises a salt of carbonic acid (such as sodium bicarbonate) in an amount of from about 5 to about 10 wt % based on the solid cosmetic product, and the third effervescent material comprises an organic acidifying agent (such as citric acid) in an amount of from about 1 to about 5 wt % based on the solid cosmetic product.

The solid cosmetic product of the present invention may also comprise one or more cosmetically acceptable additives. The person skilled in the art is aware of a range of cosmetically acceptable additives which are suitable for incorporation into such compositions. Fruit and herb extracts and juices, vegetable oils and essential oils are all compatible with the composition. Colours, both naturally derived and synthetic can be used to colour the surfactant product. Sodium carbonate may also incorporated into the composition.

In some embodiments, any one of the effervescent materials may comprise one or more cosmetically acceptable additives. In some embodiments, the cosmetically acceptable additives are selected from the group consisting of a humectant, surfactant, binder, filler, opacifier, essential oil, vitamin, perfume, fragrance, colouring, clay, vegetable butter, vegetable oil, fruit and/or herb extract, decorative item and mixtures thereof. In some embodiments the first effervescent material comprises one or more cosmetically acceptable additives selected from a humectant, surfactant, binder, filler, opacifier, essential oil, vitamin, perfume, fragrance, colouring, clay, vegetable butter, vegetable oil, fruit and/or herb extract, decorative item and mixtures thereof. In some embodiments the second effervescent material comprises one or more cosmetically acceptable additives selected from a humectant, surfactant, binder, filler, opacifier, essential oil, vitamin, perfume, fragrance, colouring, clay, vegetable butter, vegetable oil, fruit and/or herb extract, decorative item and mixtures thereof. In some embodiments the third effervescent material comprises one or more cosmetically acceptable additives selected from a humectant, surfactant, binder, filler, opacifier, essential oil, vitamin, perfume, fragrance, colouring, clay, vegetable butter, vegetable oil, fruit and/or herb extract, decorative item and mixtures thereof.

In some embodiments, any one of the effervescent materials comprises a humectant. The humectant may be selected from honey, glycerine, dipropylene glycol, monopropylene glycol and mixtures thereof. In some embodiments, any one of the effervescent materials further comprises a humectant selected from dipropylene glycol, monoproylene glycol and a mixture thereof. In some embodiments, any one of the effervescent materials comprises a humectant in an amount of from about 0.01 to about 5 wt % based on the solid cosmetic product, such as in an amount of from about 0.1 to about 1 wt % based on the solid cosmetic product, such as in an amount of from about 0.1 to about 0.5 wt % based on the solid cosmetic product. In some embodiments, the second effervescent material is the effervescent material that comprises a humectant in the above amounts, wherein the humectant is as described hereinabove.

In some embodiments, any one of the effervescent materials comprises a surfactant in an amount of from about 0.01 to about 5 wt % based on the solid cosmetic product, such as in an amount of from about 0.1 to about 1 wt % based on the solid cosmetic product, such as in an amount of from about 0.1 to about 0.5 wt % based on the solid cosmetic product. In some embodiments, the third effervescent material is the effervescent material that comprises a surfactant in the above amounts, wherein the surfactant is as described hereinabove.

Each of the first effervescent material and the second effervescent material (and third effervescent material, if present) may optionally contain colouring independently of each other. When both (or all) materials contain colouring, the colours may be selected such that a first colour in the outer material is dispersed in the water in use. When the water penetrates to the inner material a second colour may be dispersed. If this second colour is different to the first it will combine with the first to provide a change of colour to the water. In some preferred embodiments, each of the effervescent materials comprises a colouring. In some preferred embodiments, the first effervescent material comprises a different colouring to the second effervescent material.

In some embodiments, any one of the effervescent materials comprises a colouring in an amount of from about 0.01 to about 5 wt % based on the effervescent material, such as in an amount of from about 0.01 to about 3 wt % based on the effervescent material. In some embodiments, each of the effervescent materials comprises a colouring such that the total amount of colouring in the solid cosmetic product is from about 0.01 to about 5 wt % based on the solid cosmetic product, such as from about 0.01 to about 3 wt % based on the solid cosmetic product.

The essential oils will be selected based on the fragrance desired, skin type to be treated and other effects desired based on the well known properties of essential oils. The addition of essential oils, when taken in to the nose, are known to alter mood. For example, essential oils are known to create effects of drowsiness or stimulating the senses. Many well documented effects can be achieved by the use of essential oils.

In one embodiment, the one or more essential oils present in the solid cosmetic product are selected from Tarragon, Lemon myrtle, Jasmin, Ylang ylang, Labdunum, Lemongrass, Rose otto, Grapefruit, Patchouli, Rosemary, Armois, Lemon, Neroli, Sweet violet, Lavender, Orange 50 fold, Vanilla, Peppermint, Benzoin, Hydrangia, Litsea Cubeba, Cardamon, Tonka, and Chamomile blue. In one embodiment, the one or more essential oils present in the solid cosmetic product product are selected from Tarragon, Lemon myrtle, Labdunum, and Lemon.

In some embodiments, any one of the effervescent materials may comprise fragrance in an amount of from about 0.01 to about 5 wt % based on the effervescent material, such as in an amount of from about 0.1 to about 4 wt % based on the effervescent material, such as in an amount of from about 0.5 to about 2.5 wt % based on the effervescent material, such as in an amount of from about 0.5 to about 2 wt % based on the effervescent material, such as in an amount of from about 1 to about 2 wt % based on the first effervescent material. In some embodiments, the first effervescent material comprises a fragrance in an amount as described above. In some embodiments, the second effervescent material comprises a fragrance in an amount as described above. In some embodiments, the thirdeffervescent material comprises a fragrance in an amount as described above. In some preferred embodiments, each of the effervescent materials comprises a fragrance such that the total amount of fragrance in the solid cosmetic product is from about 0.01 to about 5 wt % based on the solid cosmetic product, such as from about 0.5 to about 2 wt % based on the solid cosmetic product, such as in an amount of from about 1 to about 2 wt % based on the solid cosmetic product.

Vitamins, particularly B, C and E are very beneficial for the skin. Vitamin rich ingredients such as Wheatgerm oil can also be used to deliver vitamins on to the skin. In a one embodiment, the vitamins are selected from vitamin B, vitamin C, vitamin E and mixtures thereof. It will be appreciated by one skilled in the art that the vitamin may be provided from any suitable source. For example the vitamin(s) may be provided from a synthetic source or from incorporation into the solid cosmetic product of a material, such as a natural material, that has a high vitamin content.

The ingredients in the present invention do not require cosmetic preservatives. The use of cosmetic preservatives can increase the potential to irritate the skin.

The decorative items which may be present in the solid cosmetic product include items such as glitter, paper such as rice paper, sequins, dried or fresh flowers, dried or fresh fruit, herbs, vegetables, parts thereof or mixtures thereof. Other enhancing materials such as popping candy may also be incorporated. These decorative items may be included in any one of the effervescent materials. Preferably, these decorative items are included in the second effervescent material.

The above ranges provide preferred amounts of each of the components. Each of these ranges may be taken alone or combined with one or more other component ranges to provide a preferred aspect of the invention.

### Process

As discussed herein, the invention provides a process for the production of a solid cosmetic product comprising (i) a first effervescent material, and (ii) a second effervescent material, the second effervescent material containing a salt of alginic acid, wherein each effervescent material is capable of effervescence on contact with water, wherein the rate of effervescence of the first effervescent material is greater than the rate of effervescence of the second effervescent material, wherein the first and second effervescent materials are distinct from each other, and wherein the second effervescent material envelops the first effervescent material, the process comprising the steps of:
(i) preparing the first effervescent material;
(ii) enveloping the first effervescent material with the second effervescent material.

Preferably the first effervescent material of step (i) solidifies in a predetermined shape. The first effervescent material may be caused to solidify in a predetermined shape, for example by placing the material in a mould. After the solidification of the effervescent material of step (i) the second effervescent material is placed round the solid first effervescent material so as to envelop it. The effervescent material of step (ii) also typically solidifies in a predetermined shape; for example the effervescent material of step (ii) is caused to solidify in a predetermined shape. Possible shapes include spheres, hemispheres, cubes, cuboids, pyramids and cones.

The shape of the cosmetic products of the present invention is not limited. It may be that the cosmetic products are provided with a shape which would be aesthetically pleasing and/or which aids in the use of the product. For example, it may be that the cosmetic product is produced in such a manner so that it solidifies in a shape which is ergonomically acceptable to the user. Therefore, in one embodiment of the process of the present invention, the mixture of step (i) and/or step (ii) is pressed into a mould, allowed to solidify, and then turned out to produce the cosmetic product.

As described hereinabove, the cosmetic product may further comprise a third effervescent material. In some embodiments, the process further comprises the steps of:
(iii) preparing a third effervescent material in one or more pieces; and
(iv) applying the one or more pieces of the third effervescent material to the surface of the second effervescent material.

In some embodiments, step (iii) is preparing the third effervescent material in multiple pieces, which multiple pieces are each applied to the surface of the second effervescent material in step (iv). Preferably, the one or more pieces of the third effervescent material are adhered to the outer surface of the second effervescent material. Preferably, the one or more pieces of the third effervescent material are applied to the outer surface of the second effervescent material such that the third effervescent material is not entirely covered by the second effervescent material, but the third effervescent material is at least partially embedded within the second effervescent material.

As described herein, the cosmetic product may further comprise one or more cosmetically acceptable additives. In some embodiments, the process further comprises the step of combining the mixture of step (i) and/or step (ii) with one or more cosmetically acceptable additives as defined above.

It is envisaged that the effervescent material which is enveloped need not be provided as a single discrete material. The first effervescent material which is enveloped may be present as ‘pockets’ of material which are surrounded by the second effervescent material, or the first effervescent material may be dispersed in a single mass of the second effervescent material. An analogy can be drawn with an emulsion. Thus, in one example, the first effervescent material may be provided as a number of spheres of material. These spheres of first effervescent material are enveloped by a single mass of the second effervescent material.

### Solid Cosmetic Product

In an alternative aspect, there is provided a solid cosmetic product comprising: (a) a salt of carbonic acid; (b) an organic acidifying agent; (c) a salt of alginic acid; and (d) a calcium salt.

As the skilled person will appreciate, this solid cosmetic product is an effervescent product. Preferably, the solid cosmetic product has a rate of effervescence such that the product dissolves rapidly in the water. This provides the user with an enhanced experience as the product fizzes rapidly in the water. Preferably, the solid cosmetic product has a rate of effervescence sufficient that the product travels through the water. This allows the gel formed to be dispersed throughout the water.

Therefore, in some embodiments, the solid cosmetic product has a dissolution rate at a temperature of about 37° C. (which is a typical bathing temperature) of at least about 0.1 g/sec, more preferably at least about 0.2 g/sec, more preferably at least about 0.25 g/sec, more preferably at least about 0.3 g/sec, more preferably at least about 0.35 g/sec.

In some embodiments, the salt of carbonic acid is selected from sodium bicarbonate, sodium carbonate, sodium sesquicarbonate, potassium bicarbonate, potassium carbonate, potassium sesquicarbonate, calcium bicarbonate, calcium carbonate, magnesium bicarbonate, magnesium carbonate, ammonium bicarbonate, ammonium carbonate, ammonium sesquicarbonate and mixtures thereof. In some embodiments, the salt of carbonic acid is selected from sodium bicarbonate, sodium carbonate, sodium sesquicarbonate and mixtures thereof. In some preferred embodiments, the salt of carbonic acid is sodium bicarbonate. In some preferred embodiments, the salt of carbonic acid is sodium carbonate.

In some embodiments, the organic acidifying agent is an organic acid and/or diacid. In some embodiments, the organic acidifying agent is selected from tartaric acid, cream of tartar, citric acid, fumaric acid, succinic acid, adipic acid, malic acid, maleic acid, acetic acid, oxalic acid, glutaric acid, malonic acid and mixtures thereof. In some embodiments, the organic acidifying agent is selected from tartaric acid, cream of tartar, citric acid and mixtures thereof. In some preferred embodiments, the organic acidifying agent is citric acid. In some preferred embodiments, the organic acidifying agent is cream of tartar.

In some embodiments, the salt of carbonic acid is selected from sodium bicarbonate, sodium carbonate, sodium sesquicarbonate, potassium bicarbonate, potassium carbonate, potassium sesquicarbonate, calcium bicarbonate, calcium carbonate, magnesium bicarbonate, magnesium carbonate, ammonium bicarbonate, ammonium carbonate, ammonium sesquicarbonate and mixtures thereof. Preferably, the salt of carbonic acid is sodium bicarbonate. In some embodiments, the salt of carbonic acid (such as sodium bicarbonate) is present in an amount of from about 40 to about 70 wt % based on the solid cosmetic product, such as in an amount of from about 45 to about 65 wt % based on the solid cosmetic product, such as in an amount of from about 50 to about 60 wt % based on the solid cosmetic product.

In some embodiments, the organic acidifying agent is selected from tartaric acid, cream of tartar, citric acid, fumaric acid, succinic acid, adipic acid, malic acid, maleic acid, acetic acid, oxalic acid and mixtures thereof. Preferably, the organic acidifying agent is citric acid. In some embodiments, the organic acidifying agent (such as citric acid) is present in an amount of from about 15 to about 30 wt % based on the solid cosmetic product, such as in an amount of from about 20 to about 30 wt % based on the solid cosmetic product, such as in an amount of from about 20 to about 25 wt % based on the solid cosmetic product.

In some embodiments, the salt of alginic acid is selected from sodium alginate, potassium alginate, ammonium alginate, calcium alginate, and mixtures thereof. Preferably, the salt of alginic acid is sodium alginate. In some embodiments, the salt of alginic acid (such as sodium alginate) is present in an amount of from about 0.1 to about 20 wt % based on the solid cosmetic product, such as in an amount of from about 1 to about 15 wt % based on the solid cosmetic product, such as in an amount of from about 5 to about 15 wt % based on the solid cosmetic product.

In some embodiments, the calcium salt is selected from calcium lactate, calcium chloride, calcium gluconate, calcium lactate gluconate, and mixtures thereof. Preferably, the calcium salt is calcium lactate. In some embodiments, the calcium salt (such as calcium lactate) is present in an amount of from about 0.1 to about 20 wt % based on the solid cosmetic product, such as in an amount of from about 1 to about 15 wt % based on the solid cosmetic product, such as in an amount of from about 5 to about 15 wt % based on the solid cosmetic product.

In some embodiments, the solid cosmetic product comprises the salt of alginic acid (such as sodium alginate) and calcium salt (such as calcium lactate) in a weight ratio of from about 10:1 to about 1:10, such as from about 5:1 to about 1:5, such as from about 2:1 to about 1:2, such as approximately 1:1. In some embodiments the solid cosmetic product comprises sodium alginate and calcium lactate in a weight ratio of from about 5:1 to about 1:5, such as from about 2:1 to about 1:2, such as approximately 1:1.

In some preferred embodiments, the solid cosmetic product comprises:
(a) sodium bicarbonate;
(b) citric acid;
(c) sodium alginate; and
(d) calcium lactate.

In some preferred embodiments, the solid cosmetic product comprises:
(a) a salt of carbonic acid in an amount of from about 40 to about 70 wt % based on the solid cosmetic product;
(b) an organic acidifying agent in an amount of from about 15 to about 30 wt % based on the solid cosmetic product;
(c) a salt of alginic acid in an amount of from about 0.1 to about 20 wt % based on the solid cosmetic product; and
(d) a calcium salt in an amount of from about 0.1 to about 20 wt % based on the solid cosmetic product.

In some preferred embodiments, the solid cosmetic product comprises:
(a) sodium bicarbonate in an amount of from about 40 to about 70 wt % based on the solid cosmetic product;
(b) citric acid in an amount of from about 15 to about 30 wt % based on the solid cosmetic product;
(c) sodium alginate in an amount of from about 0.1 to about 20 wt % based on the solid cosmetic product; and
(d) calcium lactate in an amount of from about 0.1 to about 20 wt % based on the solid cosmetic product.

In some embodiments, the solid cosmetic product further comprises one or more cosmetically acceptable additives. In some embodiments the one or more cosmetically acceptable additives are as described herein. In some embodiments, the one or more cosmetically acceptable additives are selected from the group consisting of a humectant, surfactant, binder, filler, opacifier, essential oil, vitamin, perfume, fragrance, colouring, clay, vegetable butter, vegetable oil, fruit and/or herb extract, decorative item and mixtures thereof. Each of these additives is as defined herein.

In some embodiments, the solid cosmetic product further comprises a humectant. The humectant may be selected from honey, glycerine, dipropylene glycol, monopropylene glycol and mixtures thereof. In some embodiments, the solid cosmetic product further comprises a humectant selected from dipropylene glycol, monoproylene glycol and a mixture thereof. In some embodiments, the solid cosmetic product further comprises a humectant (such as dipropylene glycol and/or monopropylene glycol) in an amount of from about 0.01 to about 5 wt % based on the solid cosmetic product, such as in an amount of from about 0.1 to about 2.5 wt % based on the solid cosmetic product, such as in an amount of from about 0.25 to about 1 wt % based on the solid cosmetic product, such as in an amount of from about 0.25 to about 0.75 wt % based on the solid cosmetic product.

In some embodiments, the solid cosmetic product further comprises a surfactant. The surfactant may primarily be selected from those surfactants known in the art to be suitable for contact with the skin. In some embodiments, the surfactant is selected from the group consisting of sodium laureth sulfate, cocamide diethanolamine, lauryl betaine, laureth-4 and mixtures thereof. In some embodiments, the surfactant is sodium laureth sulfate. In some embodiments, the surfactant is lauryl betaine. In some embodiments the surfactant is a mixture of sodium laureth sulfate and lauryl betaine. In some embodiments, the solid cosmetic product further comprises a surfactant (such as a mixture of sodium laureth sulfate and lauryl betaine) in an amount of from about 0.01 to about 5 wt % based on the solid cosmetic product, such as in an amount of from about 0.05 to about 2.5 wt % based on the solid cosmetic product, such as in an amount of from about 0.1 to about 1 wt % based on the solid cosmetic product.

In some embodiments, the solid cosmetic product further comprises cream of tartar in addition to the organic acidifying agent. In some embodiments, the solid cosmetic product further comprises cream of tartar in addition to the organic acidifying agent in an amount of from about 0.01 to about 5 wt % based on the solid cosmetic product, such as in an amount of from about 0.1 to about 1 wt % based on the solid cosmetic product.

The essential oils will be selected based on the fragrance desired, skin type to be treated and other effects desired based on the well known properties of essential oils. The addition of essential oils, when taken in to the nose, are known to alter mood. For example, essential oils are known to create effects of drowsiness or stimulating the senses. Many well documented effects can be achieved by the use of essential oils.

In one embodiment, the one or more essential oils present in the solid cosmetic product are selected from Tarragon, Lemon myrtle, Jasmin, Ylang ylang, Labdunum, Lemongrass, Rose otto, Grapefruit, Patchouli, Rosemary, Armois, Lemon, Neroli, Sweet violet, Lavender, Orange 50 fold, Vanilla, Peppermint, Benzoin, Hydrangia, Litsea Cubeba, Cardamon, Tonka, and Chamomile blue. In one embodiment, the one or more essential oils present in the solid cosmetic product product are selected from Tarragon, Lemon myrtle, Labdunum, and Lemon.

In some embodiments, the solid cosmetic product further comprises a fragrance in an amount of from about 0.01 to about 5 wt % based on the solid cosmetic product, such as in an amount of from about 0.1 to about 4 wt % based on the solid cosmetic product, such as in an amount of from about 0.5 to about 2 wt % based on the solid cosmetic product.

In some embodiments, the solid cosmetic product further comprises a colouring in an amount of from about 0.01 to about 5 wt % based on the solid cosmetic product, such as in an amount of from about 0.01 to about 3 wt % based on the solid cosmetic product.

Vitamins, particularly B, C and E are very beneficial for the skin. Vitamin rich ingredients such as Wheatgerm oil can also be used to deliver vitamins on to the skin. In a one embodiment, the vitamins are selected from vitamin B, vitamin C, vitamin E and mixtures thereof. It will be appreciated by one skilled in the art that the vitamin may be provided from any suitable source. For example the vitamin(s) may be provided from a synthetic source or from incorporation into the solid cosmetic product of a material, such as a natural material, that has a high vitamin content.

The ingredients in the present invention do not require cosmetic preservatives. The use of cosmetic preservatives can increase the potential to irritate the skin.

The decorative items which may be present in the solid cosmetic product include items such as glitter, paper such as rice paper, sequins, dried or fresh flowers, dried or fresh fruit, herbs, vegetables, parts thereof or mixtures thereof. Other enhancing materials such as popping candy may also be incorporated. These decorative items may be included in any one of the effervescent materials. Preferably, these decorative items are included in the second effervescent material.

In some preferred embodiments, the solid cosmetic product comprises:
(a) a salt of carbonic acid (such as sodium bicarbonate) in an amount of from about 40 to about 70 wt % based on the solid cosmetic product;
(b) an organic acidifying agent (such as citric acid) in an amount of from about 15 to about 30 wt % based on the solid cosmetic product;
(c) a salt of alginic acid (such as sodium alginate) in an amount of from about 0.1 to about 20 wt % based on the solid cosmetic product;
(d) a calcium salt (such as calcium lactate) in an amount of from about 0.1 to about 20 wt % based on the solid cosmetic product; and
(e) a fragrance in an amount of from about 0.01 to about 5 wt % based on the solid cosmetic product.

In some preferred embodiments, the solid cosmetic product comprises:
(a) a salt of carbonic acid (such as sodium bicarbonate) in an amount of from about 40 to about 70 wt % based on the solid cosmetic product;
(b) an organic acidifying agent (such as citric acid) in an amount of from about 15 to about 30 wt % based on the solid cosmetic product;
(c) a salt of alginic acid (such as sodium alginate) in an amount of from about 0.1 to about 20 wt % based on the solid cosmetic product;
(d) a calcium salt (such as calcium lactate) in an amount of from about 0.1 to about 20 wt % based on the solid cosmetic product;
(e) a fragrance in an amount of from about 0.01 to about 5 wt % based on the solid cosmetic product; and
(f) a colouring in an amount of from about 0.01 to about 5 wt % based on the solid cosmetic product.

The solid cosmetic product according to this fourth aspect of the present invention may constitute the total solid cosmetic product as described hereinabove in respect of the first aspect of the invention. In other words, the solid cosmetic product according to this fourth aspect of the present invention may constitute the total solid cosmetic product that is an aggregate of a first and second effervescent material, and optionally a third effervescent material, as described in respect of the first aspect of the invention. In such embodiment, the amounts of each of (a) a salt of carbonic acid; (b) an organic acidifying agent; (c) a salt of alginic acid; and (d) a calcium salt are as defined above in respect of this fourth aspect, and are based on the total solid cosmetic product (i.e. a combination of the first and second effervescent material, and optionally the third effervescent material).

Alternatively, the solid cosmetic product of this fourth aspect may constitute the second effervescent material as defined hereinabove in respect of the first aspect of the invention. In such embodiment, the amounts of each of (a) a salt of carbonic acid; (b) an organic acidifying agent; (c) a salt of alginic acid; and (d) a calcium salt in the solid cosmetic product are as described hereinabove in respect of the second effervescent material.

The above ranges provide preferred amounts of each of the components. Each of these ranges may be taken alone or combined with one or more other component ranges to provide a preferred aspect of the invention.

### Process

According to a further aspect of the invention, there is provided a process for the production of a solid cosmetic product comprising: (a) a salt of carbonic acid; (b) an organic acidifying agent; (c) a salt of alginic acid; and (d) a calcium salt, the process comprising the steps of:
(i) contacting the salt of carbonic acid, organic acidifying agent, salt of alginic acid and calcium salt;
(ii) solidifying the resultant composition.

In some embodiments, the solid cosmetic product solidifies in a predetermined shape. The solid cosmetic product may be caused to solidify in a predetermined shape, for example by introducing the composition into a mould. Possible shapes include spheres, hemispheres, cubes, cuboids, pyramids and cones.

The shape of the cosmetic products of the present invention is not limited. It may be that the cosmetic products are provided with a shape which would be aesthetically pleasing and/or which aids in the use of the product. For example, it may be that the cosmetic product is produced in such a manner so that it solidifies in a shape which is ergonomically acceptable to the user. Therefore, in one embodiment of the process of the present invention, the mixture of step (i) is pressed into a mould, allowed to solidify, and then turned out to produce the cosmetic product.

### Method

In one aspect of the present invention, there is provided a method comprising contacting the skin of a user with water in which the solid cosmetic product as defined herein has dissolved or in which the solid cosmetic product as defined herein is dissolving. In a typical method, water is run in to a bath at acceptable temperature. The user immerses their body in the water and the solid cosmetic product is dropped in to the water, either before or after the user immerses their body. The user may then watch the effect of the product on the surface of the water or as it effervesces beneath the surface. The user may then bathe in the water.

### EXAMPLES

The invention will now be described with reference to the following non-limiting examples.

### Example 1

A solid cosmetic product having the following composition was prepared:

### First Effervescent Material (20%):

| Raw Material Type | wt % |
|---|---|
| Sodium bicarbonate | 74.0 |
| Citric acid | 24.0 |
| Fragrance | 1.0 |
| Colour | 1.0 |
| TOTAL | 100.0 |

### Second Effervescent Material (65%):

| Raw Material Type | wt % |
|---|---|
| Sodium bicarbonate | 54.0 |
| Citric acid | 26.0 |
| Fragrance | 1.0 |
| Colour | 1.0 |
| Sodium alginate | 18.0 |
| TOTAL | 100.0 |

### Third Effervescent Material (15%):

| Raw Material Type | wt % |
|---|---|
| Sodium bicarbonate | 66.0 |
| Citric acid | 25.0 |
| Cream of tartar | 3.0 |
| Sodium laureth sulfate | 3.0 |
| Fragrance | 1.5 |
| Colour | 1.5 |
| TOTAL | 100.0 |

The solid cosmetic product was prepared as follows:
1. The first effervescent material was prepared as follows:
   a. Fragrance and colouring were dispersed through sodium bicarbonate powder
   b. Citric acid powder was contacted with the resulting mixture, and the mixture pressed into a first mould
2. The third effervescent material was prepared as follows:
   a. Sodium bicarbonate was mixed with the cream of tartar, surfactant (sodium laureth sulfate), colouring and fragrance
   b. Citric acid powder was contacted with the resulting mixture
   c. The mixture was split into multiple pieces and placed into a second mould
3. The second effervescent material was prepared as follows:
   a. Sodium bicarbonate was mixed with the colouring, fragrance and sodium alginate
   b. Citric acid powder was contacted with the resulting mixture
4. The first effervescent material was then removed from the mould and the second effervescent material as prepared was pressed around it and place into the second mould with the pieces of third effervescent material already contained therein.
5. When the product had hardened, it was removed from the mould.

### Example 2

### First Effervescent Material (22.5%):

| Raw Material Type | wt % |
|---|---|
| Sodium Bicarbonate | 74.0 |
| Citric acid | 24.0 |
| Colour | 1 |
| Fragrance | 1 |
| TOTAL | 100.0 |

### Second Effervescent Material (67.5%):

| Raw Material Type | wt % |
|---|---|
| Sodium Bicarbonate | 54.0 |
| Citric acid | 26.0 |
| Colour | 1.25 |
| Fragrance | 1.25 |
| Sodium Alginate | 17.0 |
| Dried fruit | 0.5 |
| TOTAL | 100.0 |

### Third Effervescent Material (10%):

| Raw Material Type | wt % |
|---|---|
| Sodium Bicarbonate | 74.0 |
| Citric acid | 24.0 |
| Colour | 1 |
| Fragrance | 1 |
| TOTAL | 100.0 |

The method for preparing the product was as recited above for Example 1.

### Example 3

### First Effervescent Material (25%):

| Raw Material Type | wt % |
|---|---|
| Sodium Bicarbonate | 75.0 |
| Citric acid | 24.0 |
| Colour | 0.5 |
| Fragrance | 0.5 |
| TOTAL | 100.0 |

### Second Effervescent Material (60%):

| Raw Material Type | wt % |
|---|---|
| Sodium Bicarbonate | 54.0 |
| Citric acid | 26.0 |
| Fragrance | 1.0 |
| Colour | 1.0 |
| Sodium Alginate | 9.0 |
| Calcium Lactate | 9.0 |
| TOTAL | 100.0 |

### Third Effervescent Material (15%):

| Raw Material Type | wt % |
|---|---|
| Sodium Bicarbonate | 66.5 |
| Citric acid | 25.0 |
| Cream of Tartar | 2.0 |
| Sodium Laureth Sulfate | 1.0 |
| Lauryl Betaine | 1.0 |
| Fragrance | 2.0 |
| Colour | 2.5 |
| TOTAL | 100.0 |

The method for preparing the product was as recited above for Example 1.

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

1. A solid cosmetic product comprising:
(i) a first effervescent material, and
(ii) a second effervescent material, the second effervescent material containing a salt of alginic acid,
wherein each effervescent material is capable of effervescence on contact with water,
wherein a rate of effervescence of the first effervescent material is greater than a rate of effervescence of the second effervescent material,
wherein the first effervescent material and the second effervescent material are distinct from each other, and wherein the second effervescent material envelops the first effervescent material.

2. The solid cosmetic product according to claim 1, wherein the second effervescent material entirely envelops the first effervescent material.

3. The solid cosmetic product according to claim 1, wherein the salt of alginic acid is selected from sodium alginate, potassium alginate, calcium alginate, and mixtures thereof.

4. The solid cosmetic product according to claim 1, wherein the second effervescent material comprises the salt of alginic acid in an amount of from about 5 wt % to about 25 wt % based on the second effervescent material.

5. The solid cosmetic product according to claim 1, wherein the first effervescent material and the second effervescent material each comprises a salt of carbonic acid and an organic acidifying agent.

6. The solid cosmetic product according to claim 5, wherein the salt of carbonic acid is selected from sodium bicarbonate, sodium carbonate, sodium sesquicarbonate and mixtures thereof.

7. The solid cosmetic product according to claim 5, wherein the organic acidifying agent is selected from tartaric acid, cream of tartar, citric acid and mixtures thereof.

8. The solid cosmetic product according to claim 1, wherein the first effervescent material comprises a salt of carbonic acid in an amount of from about 50 wt % to about 80 wt % based on the first effervescent material, and an organic acidifying agent in an amount of from about 15 wt % to about 30 wt % based on the first effervescent material.

9. The solid cosmetic product according to claim 1, wherein the second effervescent material comprises a salt of carbonic acid in an amount of from about 35 wt % to about 60 wt % based on the second effervescent material, and an organic acidifying agent in an amount of from about 15 wt % to about 30 wt % based on the second effervescent material.

10. The solid cosmetic product according to claim 1, wherein the first effervescent material is present in an amount of from about 10 wt % to about 60 wt % based on the solid cosmetic product.

11. The solid cosmetic product according to claim 1, wherein the second effervescent material is present in an amount of from about 20 wt % to about 85 wt % based on the solid cosmetic product.

12. The solid cosmetic composition according to claim 1, further comprising a third effervescent material capable of effervescence on contact with water, wherein the third effervescent material is distinct from the first effervescent material and the second effervescent material.

13. The solid cosmetic product according to claim 12, wherein the third effervescent material is in one or more distinct pieces disposed at a surface of the second effervescent material.

14. The solid cosmetic product according to claim 12, wherein the rate of effervescence of the second effervescent material is greater than a rate of effervescence of the third effervescent material.

15. The solid cosmetic product according to claim 12, wherein the rate of effervescence of the third effervescent material is greater than a rate of effervescence of the second effervescent material.

16. The solid cosmetic product according to claim 12, wherein the third effervescent material is present in an amount of from about 1 wt % to about 30 wt % based on the solid cosmetic product.

17. The solid cosmetic product according to claim 1, wherein any one of the effervescent materials comprises a calcium salt.

18. The solid cosmetic product according to claim 17, wherein the calcium salt is selected from calcium lactate, calcium chloride, calcium gluconate, calcium lactate gluconate, and mixtures thereof.

19. The solid cosmetic product according to claim 17, wherein the calcium salt is present in an amount of from about 5 wt % to about 15 wt % based on the effervescent material.

20. The solid cosmetic product according to claim 17, wherein the calcium salt is present in the second effervescent material.

21. The solid cosmetic product according to claim 1, wherein the solid cosmetic product has a density of less than 1 g/cm³.

22. The solid cosmetic product according to claim 1, wherein any one of the effervescent materials comprises at least one component selected from a humectant, surfactant, binder, filler, opacifier, essential oil, vitamin, perfume, fragrance, colouring, clay, vegetable butter, vegetable oil, fruit and/or herb extract, decorative item and mixtures thereof.

23. A process for the production of a solid cosmetic product as defined in claim 1, the process comprising the steps of:
(i) preparing the first effervescent material;
(ii) enveloping the first effervescent material with the second effervescent material.

24. The process according to claim 23, wherein the first effervescent material and/or the second effervescent material is caused to solidify in a predetermined shape.

25. The process according to claim 23, further comprising the steps of:
(iii) preparing a third effervescent material in one or more pieces; and
(iv) applying the one or more pieces of the third effervescent material to a surface of the second effervescent material.

26. A product obtained or obtainable by the process of claim 23.

27. A solid cosmetic product comprising:
(a) a salt of carbonic acid;
(b) an organic acidifying agent;
(c) a salt of alginic acid; and
(d) a calcium salt.

28. The solid cosmetic product according to claim 27, wherein the salt of carbonic acid is selected from sodium bicarbonate, sodium carbonate, sodium sesquicarbonate, and mixtures thereof.

29. The solid cosmetic product according to claim 27, wherein the organic acidifying agent is selected from tartaric acid, cream of tartar, citric acid and mixtures thereof.

30. The solid cosmetic product according to claim 27, wherein the salt of alginic acid is selected from sodium alginate, potassium alginate, calcium alginate, and mixtures thereof.

31. The solid cosmetic product according to claim 27, wherein the calcium salt is selected from calcium lactate, calcium chloride, calcium gluconate, calcium lactate gluconate, and mixtures thereof.

32. The solid cosmetic product according to claim 27, wherein the salt of alginic acid and the calcium salt are present in a weight ratio of from about 5:1 to about 1:5.

33-34. (canceled)
